(19) United States
(12) Patent Application Publication
(10) Pub. No.: US 2023/0181000 A1
(43) Pub. Date: Jun. 15, 2023

(54) METHOD AND SYSTEM FOR IMAGE-GUIDED PROCEDURES WITH SENSING STYLET

(71) Applicant: Andrei Vertikov, Westwood, MA (US)

(72) Inventor: Andrei Vertikov, Westwood, MA (US)

(21) Appl. No.: 17/990,673

(22) Filed: Nov. 19, 2022

Related U.S. Application Data
(60) Provisional application No. 63/281,913, filed on Nov. 22, 2021.

(51) Int. Cl. A61B 1/00; A61B 10/04; A61B 1/07

(52) U.S. Cl. CPC A61B 1/000094; A61B 10/04; A61B 1/00183; A61B 1/0002; A61B 1/00195; A61B 1/07; A61B 1/0005; A61B 2010/045

(57) ABSTRACT

A medical apparatus includes: an endoscopic subsystem, a medical instrument, an imaging stylet; and a system console with data-processing capability. This image-guided system calculates, in-real time, a position of the instrument relative to a target within patient body to guide and control accurate placement of the instrument to the target. The stylet is configured to acquire image data intra-operatively. In addition, the stylet has a sensing region along a flexible distal portion of its length. The system console communicates with the stylet to calculate the position of the instrument inside a patient by using intra-operative image data of surrounding tissue acquired by the stylet, distributed strain data measured by the console within the sensing region of the stylet, and preoperative image data of the patient anatomy. The stylet incorporates optical guides that are advantageously used both for imaging and for distributed strain sensing, enabling miniaturization of the stylet for accomplishing an intra-operative image guidance and navigational feedback without increasing invasiveness or compromising safety of the guided medical procedures.

### CROSS-REFERENCE TO RELATED APPLICATIONS

This application claims priority to and the benefit of U.S. provisional patent application Ser. No. 63/281,913 filed on Nov. 22, 2021 entitled “Method and System For Image-guided Procedures with Sensing Stylet”, the entire disclosure of which is hereby incorporated by reference as if set forth in its entirety for all purposes.

### TECHNICAL FIELD

The present invention relates to the field of diagnostic medical imaging and image guidance for medical procedures and, more specifically, to minimally invasive image-guided procedures in luminal anatomic structures, naturally or surgically created body cavities, or interstitially in tissue.

### BACKGROUND

Minimally-invasive procedures. The need for effective imaging for medical diagnostics and for guidance and control of diagnostic, therapeutic and surgical procedures is well recognized. Often such imaging has to be performed in a complex network of narrow and difficult-to-reach body lumens (such as, for example, blood vessels of cardiovascular and neurovascular systems, airway tree of lungs, gastrointestinal, bile and urinary tracts) or in tight spaces of natural or surgically created body cavities. In general terms, the image guidance is aimed at identifying and localizing a specific target within patient body and then accurately placing a medical tool in this target while avoiding anatomical risk structures. Many medical tools, exist with specific intended uses for specific medical procedures. For example, the medical tool can be an instrument, such as an injection needle, that delivers therapeutic agents. The medical tool can be a biopsy instrument, such as an aspiration needle or biopsy forceps, that takes tissue samples. The medical tool can be also a surgical instrument for diseased tissue resection or a probe that deposits RF, MW, laser and alike energy or cryogenically cools the tissue for diseased tissue ablation. There exist flexible and rigid endoscopic imaging devices, for example bronchoscopes, thoracoscopes, laparoscopes and alike, that address the need for minimally-invasive image guided procedures utilizing various medical tools. Some endoscopes incorporate small cameras and illumination fibers at their distal ends together with working channels for deployment of endoscopic medical instruments under visual guidance. Other endoscopes are configured to work with surgical instruments that are deployed via separate incisions or surgically created openings.

Multi-modality. Often several imaging modalities need to be advantageously combined for effective image guidance. For example, target tissue region can be located deeper under observed surface and thus cannot be easily visualized with standard endoscopy. In this case, subsurface imaging modalities such as, for example, endoscopic ultrasound might help localize and guide tools to a target. Accordingly, there exist endoscopes with integrated ultrasound imaging or with ability to accept endoscopic ultrasound probes. For example there are flexible endobronchial ultrasound probes (EBUS) for use in bronchoscopic image guided procedures. There are also flexible endoscopes with integrated linear EBUS imaging to visualize and guide medical tools [Ref 1]. More recently, flexible Optical Coherence Tomography (OCT) imaging probes have been developed for sub-surface imaging [Ref 2]. OCT imaging has advantage of high resolution when compared to ultrasound. However, integration of additional modalities increases dimensions of distal ends of endoscopes thus limiting their use in smaller lumens or cavities. On the other hand, the use of separate imaging probes in endoscopically guided procedures may require an exchange of tools that share the same working channel. Such a tool exchange increases durations of medical procedures and thus increases patient distress; the exchange also affects accuracy of the tool placement to a target.

Pre-operative imaging, instrument navigation, and instrument performance. Many medical procedures start with a planning phase that uses preoperative, or pre-procedural, image data, for example obtained with CT or MRI, to construct tool trajectory for deployment to a target. The tool position on the planned trajectory needs to be tracked, however, for accurate navigation to the target. Significant challenges exist for tracking of flexible endoscopes and flexible medical instruments especially in soft tissue when tissue can deform and organs can shift [Ref 3]. While the use of localization elements such as EM position and orientation sensors integrated in endoscopes and/or in medical instruments are known in the field of medical instrument navigation, added dimensions of the localization elements limit functionality of EM-guided instruments, Generally, there is a trade-off between miniaturization and performance of a medical tool. For example, biopsy accuracy depends on amount of sample tissue collected and this amount is proportional to a cross-sectional area of internal lumen of a sample collecting instrument. Overall, there is a need for miniaturized image-guided medical instruments that can be accurately navigated in the tight spaces of minimally invasive procedures without compromising their performance and safety.

The present invention is intended to address these and several other deficiencies of minimally invasive image-guided procedures as described below.

### SUMMARY OF OBJECTIVES AND EXEMPLARY EMBODIMENTS

Embodiments of the invention provide an image-guided system that includes: an endoscopic subsystem, a medical instrument, an imaging stylet; and a system console with data-processing capability. This image-guided system calculates, in-real time, a position of the instrument relative to a target within patient body. This position is used to guide and control accurate placement of the instrument to the target.

Main objective of the present invention is to provide an intra-operative image guidance and navigational feedback for minimally-invasive medical procedures without increasing dimensions or compromising safety and efficacy of navigated medical instruments. Accordingly, in specific embodiments, a miniaturized imaging probe in a form of a flexible stylet is provided. The stylet is insertable in a lumen of a therapeutic, a diagnostic, a surgical, or a tissue marking endoscopic medical instrument; with an arrangement of the imaging stylet and the instrument configured to acquire image data intra-operatively. In addition, the stylet has a sensing region along a flexible distal portion of its length. A system console is also provided that communicates with the stylet to calculate the position of the instrument inside a patient by using intra-operative image data of surrounding tissue acquired by the stylet, distributed strain data measured by the console within the sensing region of the stylet, and pre-operative image data of the patient anatomy. The stylet incorporates, as a main aspect of the invention, optical guides that are advantageously used both for imaging and for distributed strain sensing, enabling miniaturization of the stylet for accomplishing the main objective of the invention.

In one embodiment, the imaging stylet incorporates an eccentric rotatable guide of optical energy that couples proximal and distal ends of the stylet; the said rotatable optical guide is disposed within the stylet body with a lateral offset relative to the rotational axis of the guide and also relative to the neutral bending axis of the stylet. In operation, the optical guide rotates freely within the stylet to generate distal scanned patterns for tissue imaging using optical elements attached to the optical guide distally. At the same time, the system console measures, within a distal sensing region of the same rotating optical guide, a spatial distribution of time-varying strain modulated by the said rotation. The stylet or the instrument position is then calculated by analyzing intra-operatively acquired image data, intra-operatively measured strain distribution data, and pre-operative image data of patient anatomy.

Other embodiments provide structures within the imaging stylet that incorporate eccentric optical guides fixedly attached to the stylet body with lateral offsets relative to the neutral bending axis of the stylet. In this embodiment, each individual eccentric optical guide directs, using distal optics at the tip of the stylet, a portion of optical energy towards an imaged tissue thus forming an optical beam with a fixed spatial relationship with the stylet distal end. At least in some embodiments, a single distal optical element directs optical energy towards an imaged tissue from a plurality of the fixed eccentrically-positioned optical guides. In some specific embodiments, the said single directing element is a curved mirror or a faceted mirror. Yet in other specific embodiments, the single directing optical element is a wide-angle refractive lens or a diffractive metalens. In operation, the system console acquires one-dimensional (1D) image data sets from the individual fixed optical beams outcoupled from the corresponding optical guides and also measures strain distributions within sensing regions of at least some eccentrically positioned optical guides. The stylet or the instrument position is then calculated by analyzing intra-operatively acquired 1D image data sets, intra-operatively measured strain distribution data, and pre-operative image data of patient anatomy. During repositioning of the stylet, the system console combines the acquired 1D image data sets, remapping the said 1D image data using the stylet position information to render 2D or 3D scenes of imaged tissue with extended fields of view.

Some other embodiments provide structures within the imaging stylet that integrate eccentrically positioned optical guides with a distal scanning mechanism that generates scanned patterns of optical energy emitted by the stylet towards an image tissue. In some specific embodiments, a piezo element is disposed distally between the optical waveguides and a distal optics of the stylet to actuate X-Y scanning of a distal tip of an optical guide. In related embodiments, a stepped outer diameter structure of the distal end of an optical energy guide is provided to facilitate integration of the optical guide and a distal scanning arrangement in a miniaturized stylet. Additionally, at least in some related embodiments, a concentrating optical element is disposed between eccentric optical energy guides and a distal optics of the stylet to improve the collection efficiency for optical energy returned by an imaged tissue. Yet, in some other specific embodiments, a torsional scanning arrangement is provided disposed distally within the stylet body. The said torsional scanning arrangement rotationally reciprocate distal ends of eccentrically-positioned optical guides to generate oscillating scanned patterns of optical energy outcoupled by the stylet for tissue imaging. In some related embodiments, a tubular structure with deposited coiled electrodes in operable communication with the console and a portion of an optical guide coated with a magnetic material form an electro-magnetically actuated torsional scanning arrangement. Additionally, at least in some related embodiments, the single directing optical element mentioned above forms beams of optical energy outcoupled towards a tissue from a plurality of eccentric optical guides scanned by a distal torsional scanning arrangement. The stylet or the instrument position is then calculated by analyzing intra-operatively acquired image data, intra-operatively measured strain distribution data, and pre-operative image data of patient anatomy. In some embodiments, the system console remaps and re-renders intra-operatively acquired image data in accordance with the calculated stylet positions.

Methods of using the image-guided system of the present inventions to address the above objectives are also provided.

### BRIEF DESCRIPTION

The invention will be more fully understood by referring to the following Detailed Description in conjunction with the Drawings, of which:

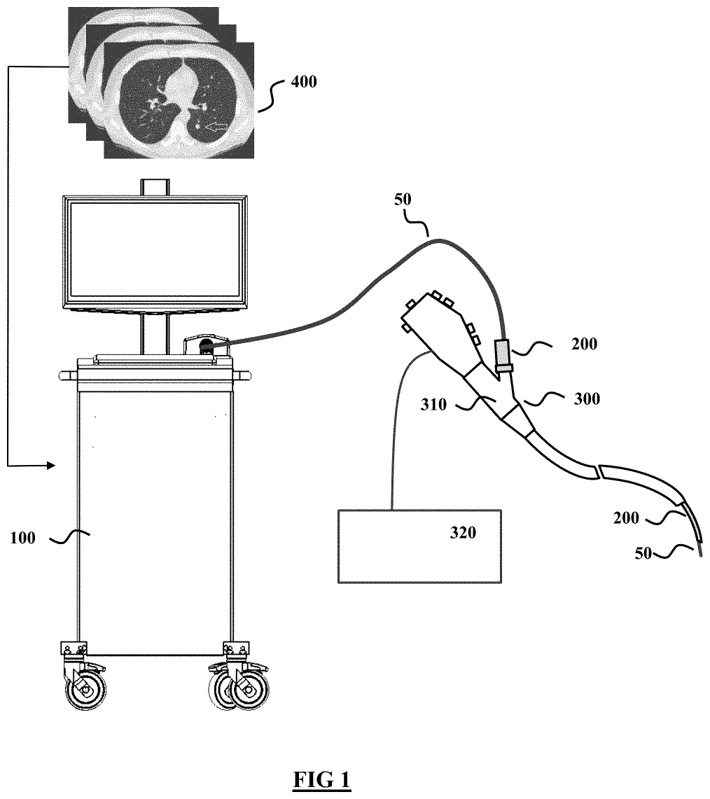

FIG. 1 is a top view of a medical system of the invention;

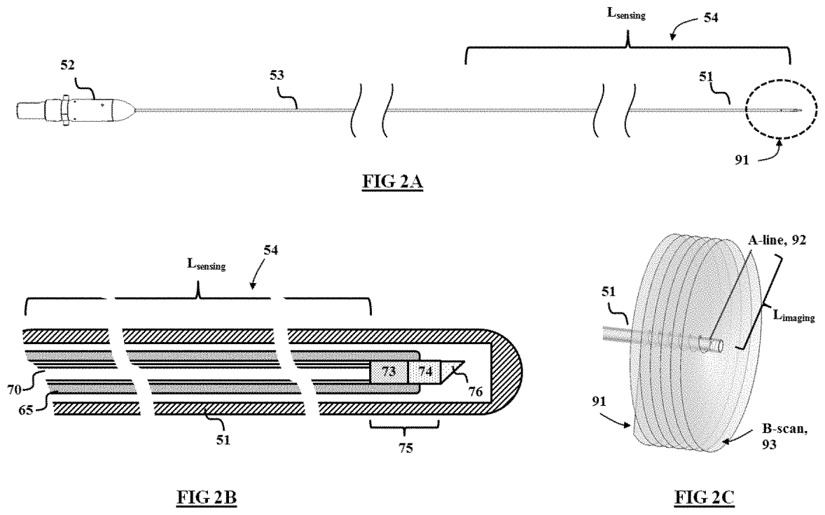

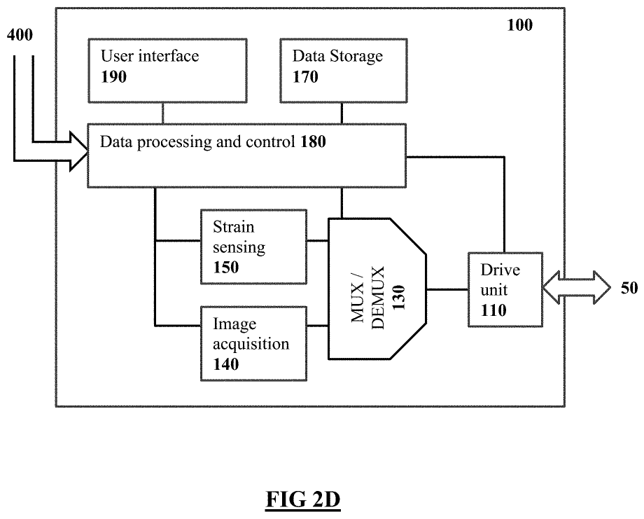

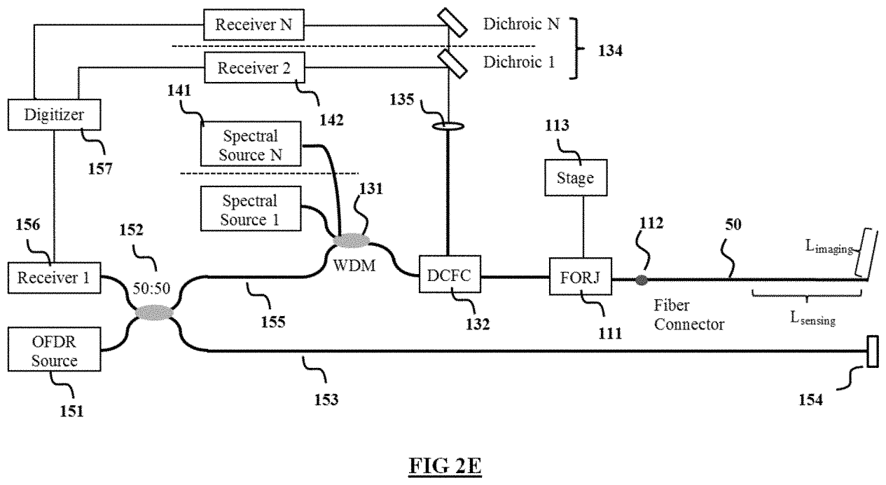

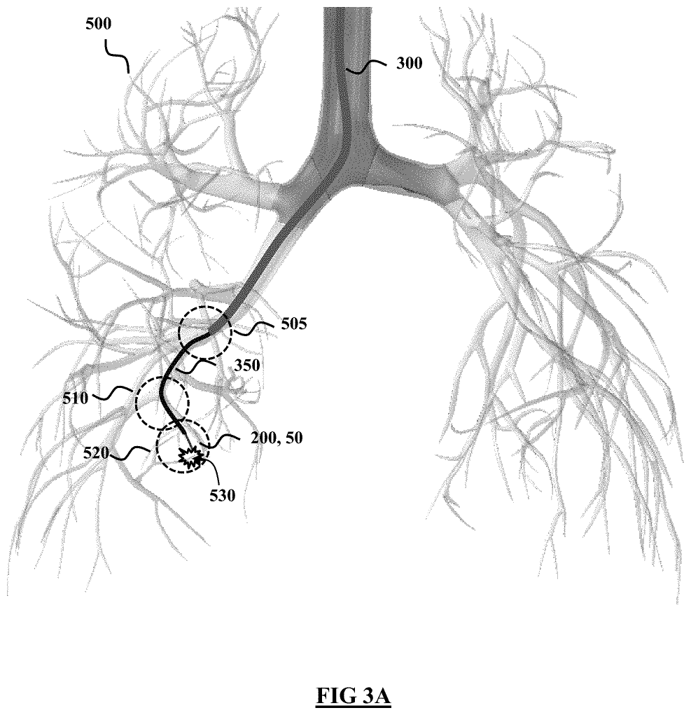

FIG. 2A is a top view of an imaging stylet of the invention, with FIG. 2B showing a partial section of a distal end of the stylet in one specific embodiment and FIG. 2C illustrating a volume imaged by the stylet in one specific embodiment;

FIG. 2D is a block diagram of a system console of the invention, with FIG. 2E showing strain-sensing and imaging arrangements of the system console in accordance with one embodiment of the invention;

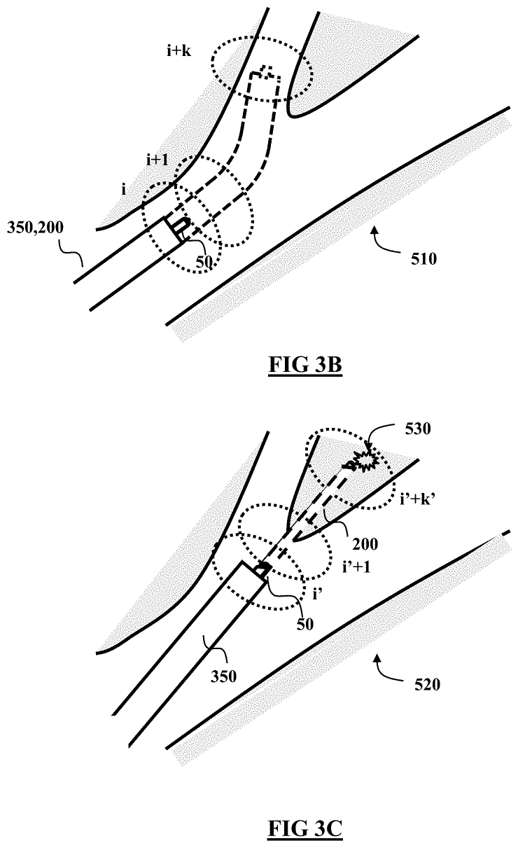

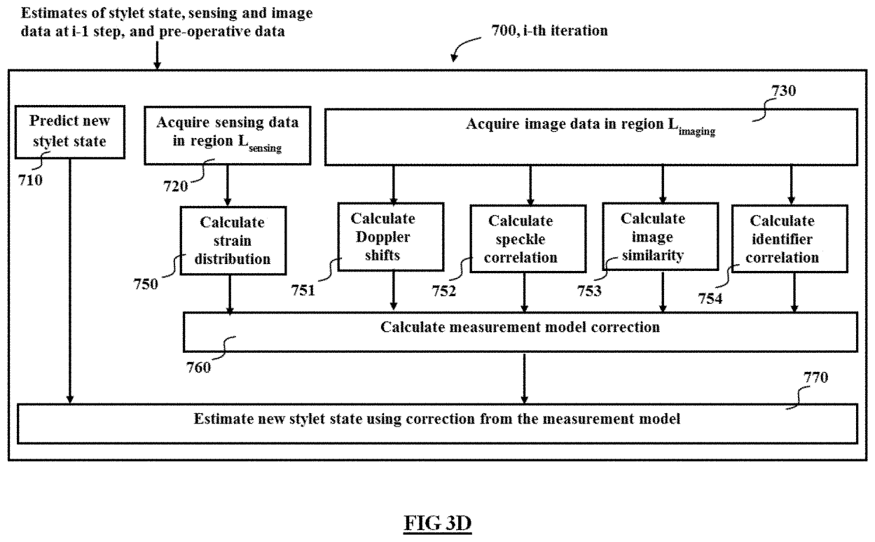

FIG. 3A is a front view of an airway tree of a patient with the system of the invention, wherein an endoscopic instrument with the imaging stylet of the inventions is navigated endobronchially and transbronchially towards a target tissue;

FIGS. 3B and 3C are detail views of endobronchial and transbronchial portions of navigation, respectively, of the endoscopic instrument towards the target in accordance with the method of the invention;

FIG. 3D is a flow chart of calculating position of the imaging stylet using fusion of distributed strain sensing and image data acquired by the stylet in accordance with the method of the invention;

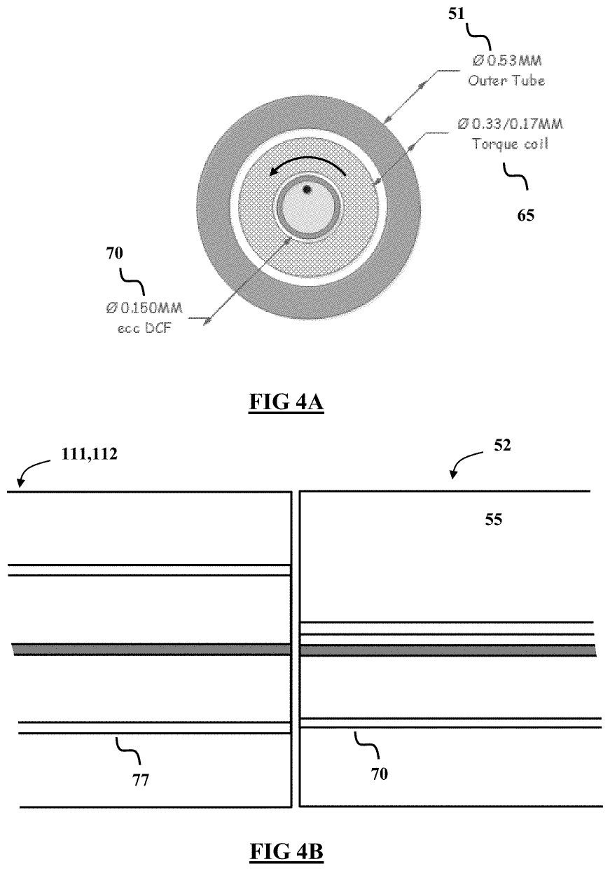

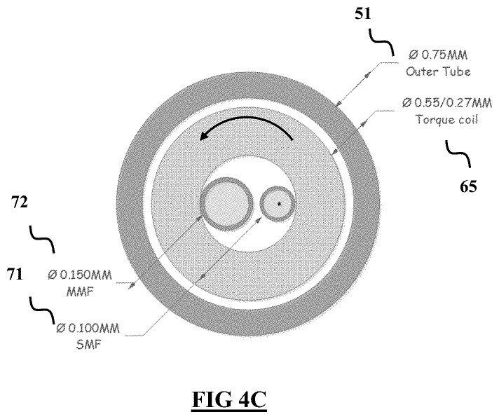

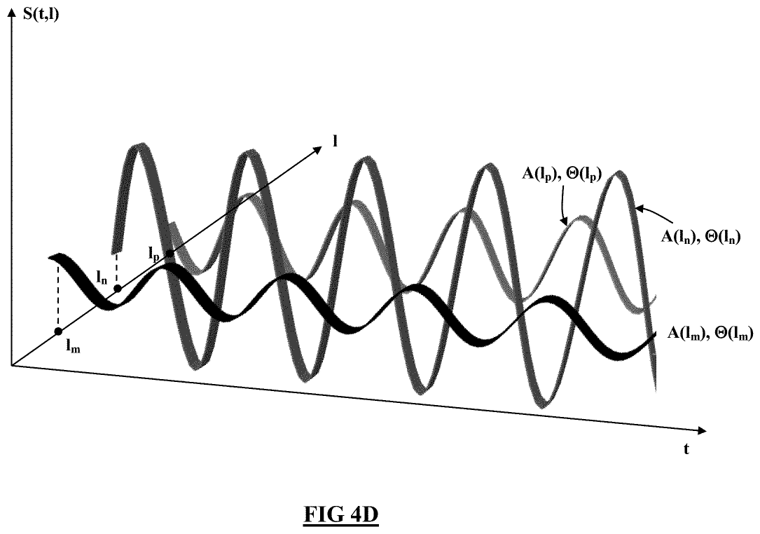

FIG. 4A is a partial section of the imaging stylet in its sensing region that shows a rotatable strain-sensing optical channel in accordance with one example of the first preferred embodiment of the invention;

FIG. 4B shows a partial section of a corresponding proximal end of the stylet interfacing optically with the system console in accordance with the same example the first preferred embodiment of the invention;

FIG. 4C is a partial section of the imaging stylet in its sensing region that shows a rotatable strain-sensing optical channel in accordance with second example of the first preferred embodiment of the invention;

FIG. 4D illustrates exemplary spatial and temporal distributions of strain signals in a rotating strain-sensing optical channel of the imaging stylet in accordance with the first preferred embodiment of the invention;

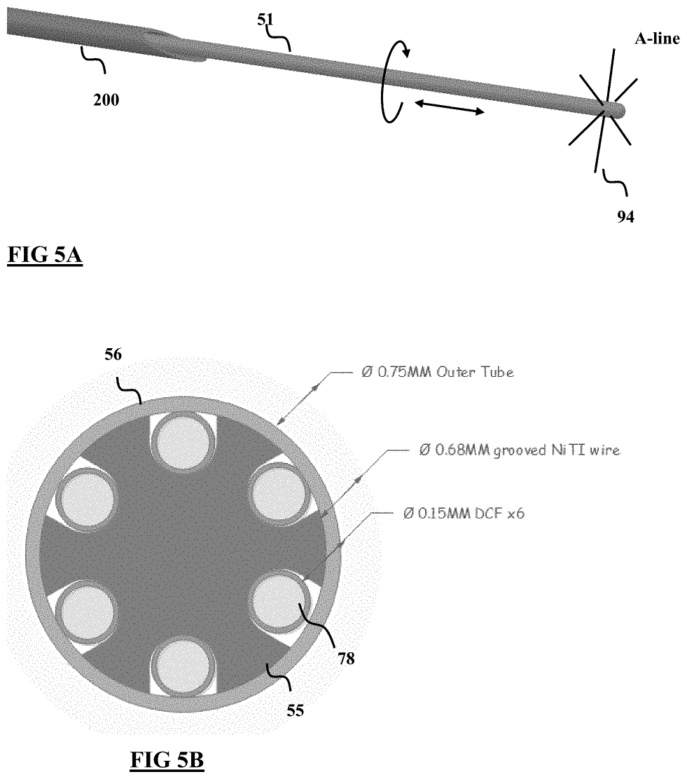

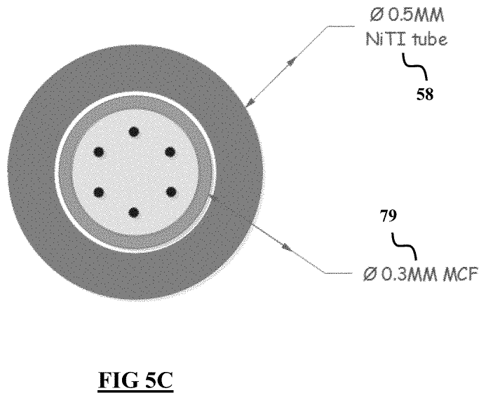

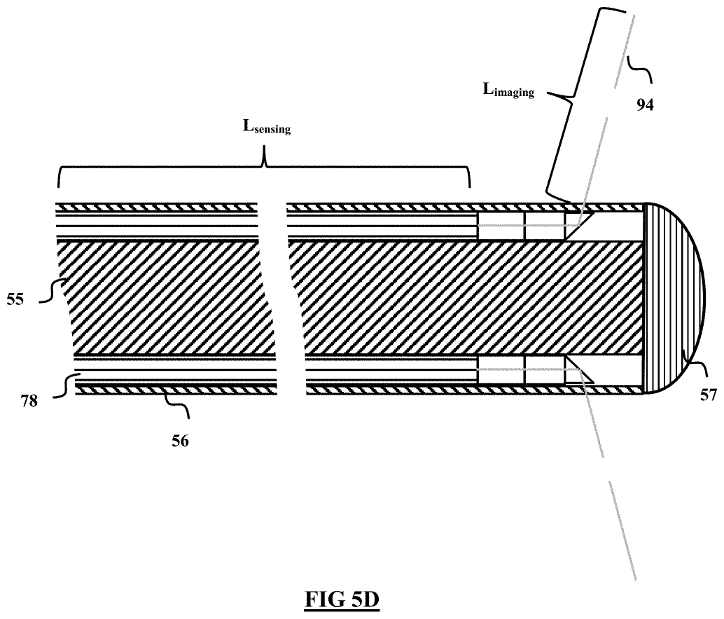

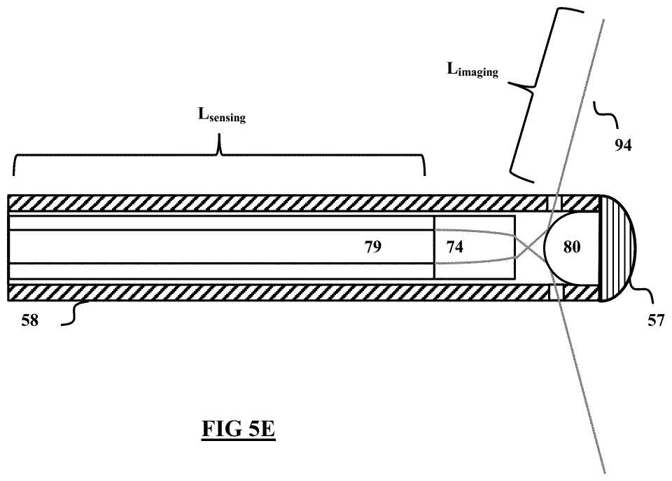

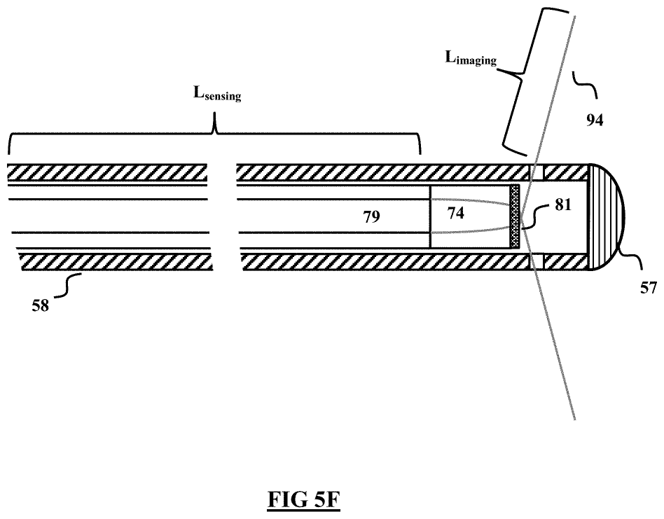

FIG. 5A shows a top view of the distal end of the imaging stylet inserted in an endoscopic needle in accordance with the second preferred embodiment of the invention;

FIGS. 5B-5F are partial sectional views of exemplary strain-sensing and imaging arrangements in the distal end of the imaging stylet in accordance with the second preferred embodiment of the invention;

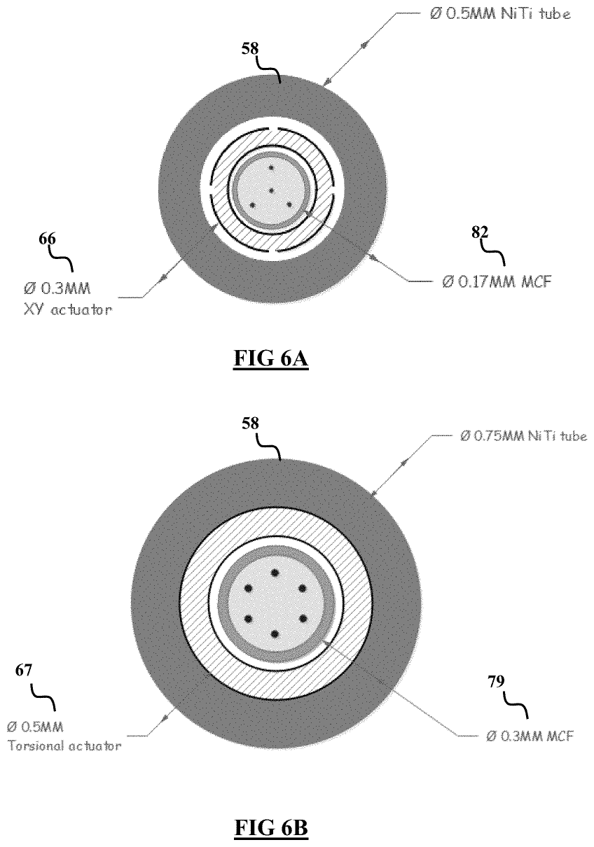

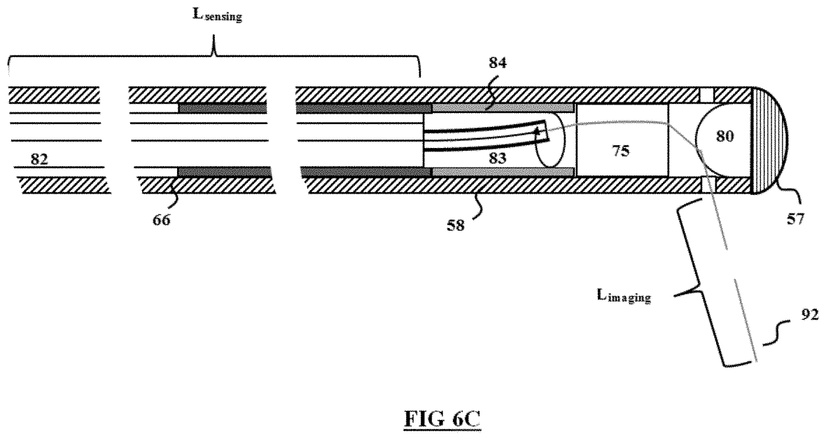

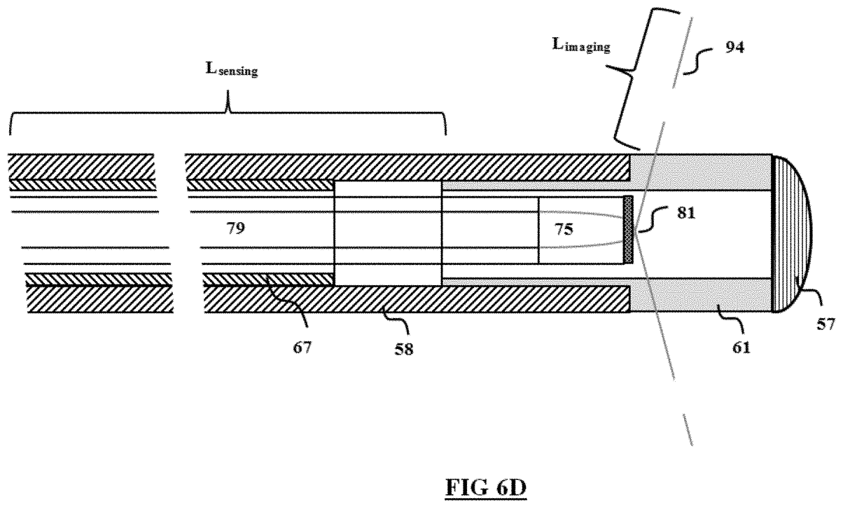

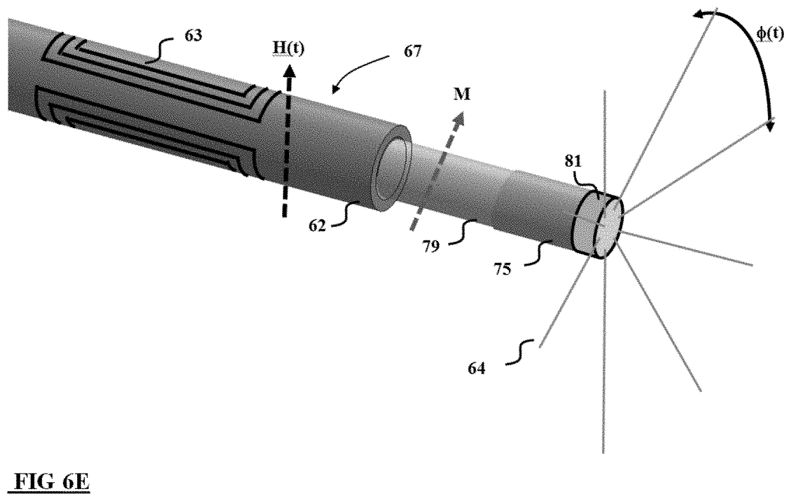

FIGS. 6A-6D are partial sectional views of exemplary strain-sensing and imaging arrangements in the distal end of the imaging stylet in accordance with the third preferred embodiment of the invention, wherein FIG. 6E shows an exemplary torsional scanning arrangement in accordance with this embodiment;

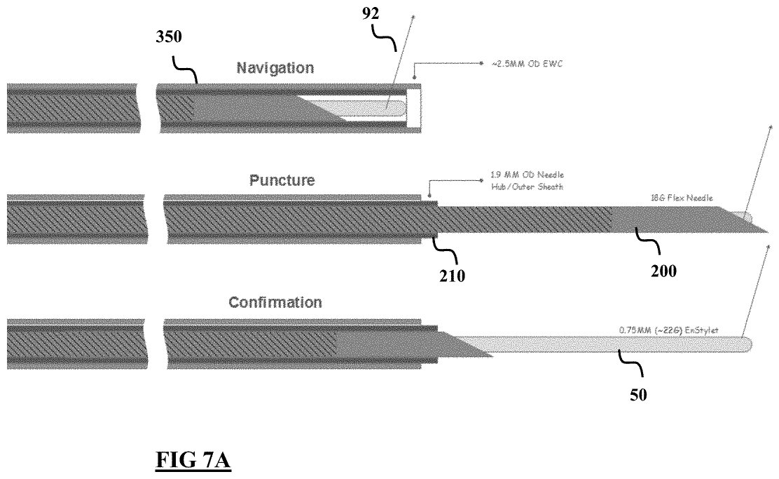

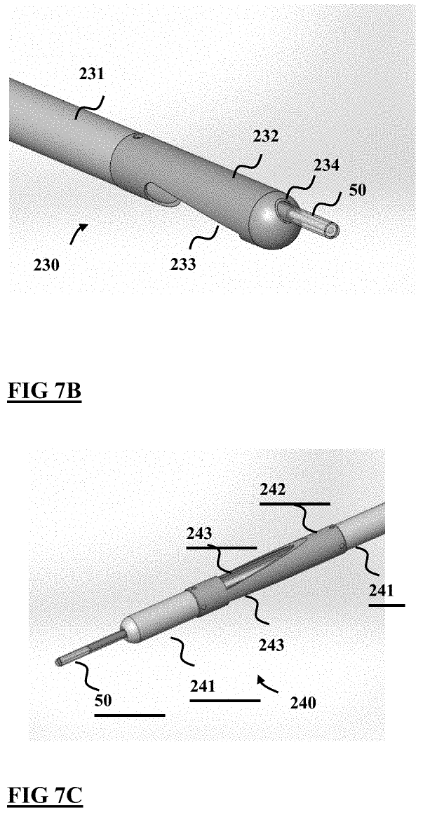

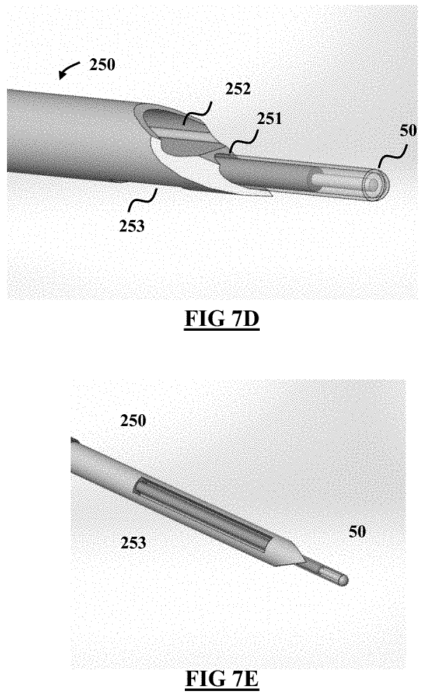

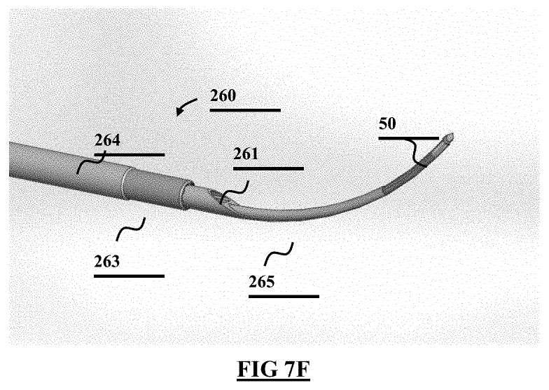

FIG. 7A illustrates steps of navigating an endoscopic biopsy needle to the target tissue using the method of the invention, while FIGS. 7B-7F show other exemplary endoscopic instruments that can be navigated to the target tissue in accordance with the method of invention.

### DETAILED DESCRIPTION

For clarity of the presentation, the following disclosure is structured subdivided as follows. The description associated with FIGS. 1 through 3 is the general description of the medical apparatus and the methods of the present invention. FIGS. 4, 5, and 6 relate to the first, the second, and the third preferred embodiments of the present invention, respectively. Finally, FIG. 7 describe an exemplary medical procedure and associated medical instruments used with the method and the system of the present invention.

### General Description.

FIG. 1 shows an exemplary embodiment of the medical system of the invention. The system has a flexible imaging probe 50 also referred to as an imaging stylet in this disclosure. The stylet 50 is configured to acquire image data intra-operatively. It is also configured to sense strain distribution in at least one portion of its elongated body. The system also contains an endoscopic sub-system 300 comprising an endoscope 310 and an endoscope control and display unit 320. In addition, the system includes a medical instrument 200 deployable via a working channel of the endoscope 310. The distal end of the imaging stylet is configured to be insertable in a lumen of the endoscopic medical instrument 200. During operation, the imaging stylet 50 is in communication at its corresponding proximal ends with a system console 100 that processes intra-operative image data and strain sensing data received from the stylet 50. Using the intra-operative data together with pre-operative image data 400, the system console 100 calculates a position of the medical instrument 200 or the stylet 50 relative to a target in the patient body. Aggregately, the stylet 50, the medical instrument 200, the endoscopic sub-system 300, and the system console 100 are referred to as a medical system or a medical apparatus of the present invention.

The term “distal ends” implies, in the context of the present disclosure, distal end portions of medical instruments intended to be placed inside or in close proximity to the patient body lumens, cavities, tissue and other targets for a medical procedure. The term “proximal ends” implies, in the context of the present disclosure, the corresponding “opposite” portions of the medical instruments that are intended to be held and manipulated by an operator or to be interfaced with the system console 100. The term “medical tool” or “medical instrument” implies, in the context of the present disclosure, any interventional diagnostic, treatment, or marking medical instrument or medical device. Non-limiting examples of the medical tools of the present invention are: an injection needle, an aspiration needle, core biopsy needle, side cutting needles, biopsy or cutting forceps, snatches, fiducials placement devices, stent placement devices, balloons, surgical cutters, RF, MW, laser, cryogenic ablation devices, photodynamic therapy delivering devices. The term endoscope implies, in the context of the present disclosure, any flexible or rigid endoscopic imaging device or system such as a bronchoscope, a laparoscope, a surgical robotic system, an endoscopic robotic system and alike. Accordingly, medical instruments imply, in the context of the present disclosure, both endoscopic instruments deployable via endoscopic working channels and surgical instruments deployable via separate surgical ports.

The terms “position”, “probe position”, “stylet position”, and “instrument position” imply, in the context of this disclosure, both a position of the stylet or positions of associated medical tools of the invention, unless the context clearly dictates otherwise. Also, the term “position” implies both a position and an angular orientation of the stylet distal end, unless the context clearly dictates otherwise. In addition, the term “or” is an inclusive “or” operator and is equivalent to the term “and/or”, unless the context clearly dictates otherwise. The term “real-time” implies, in the context of the present disclosure, substantially real-time, that is sufficiently fast so that an alignment of a probe or a tool relative to a target is not lost due to uncontrolled motion.

The term “intra-operative data” and related terms imply, in the context of this disclosure, image data and strain-sensing data acquired by the imaging stylet of the invention. The term “pre-operative data” and related terms imply either image data acquired by other imaging devices such as, for example, X-ray, CT, MRI, OCT, or ultrasound devices or image data acquired by the imaging probe of the invention in a previous medical procedure or both, unless the context clearly dictates otherwise.

Referring now to FIG. 2A, the stylet 50 is an elongated flexible body characterized by its longitudinal axis, the stylet consisting of a distal end 51 and a proximal end 52 that are connected with a flexible shaft 53. When in operable communication with the device console, the stylet 50 projects an interrogating optical energy (understood to be UV, visible or NIR optical radiation with wavelengths in the range 0.2-2 μm) at its distal end 51 toward the ambient medium that may include an interrogated tissue. The stylet 50 also receives a returned optical energy originated at the tissue in response to the interrogating energy, the returned energy being encoded by the tissue response in an imaged region 91. Corresponding image data that contains information about structure, composition, or function of the tissue in the region 91 is then obtained using interferometric methods known as Optical Coherence Tomography (OCT). The methods of OCT that are described in References 2 and References 4-7 can be applied for the purpose of the present invention and are incorporated by reference herein in their entirety. Alternatively or in addition, other spectral information from the region 91 can be obtained with some embodiments of the invention. This additional spectral information can be obtained with reflectance spectroscopy, absorption spectroscopy, Raman spectroscopy, fluorescence spectroscopy and time-resolved fluorescence as described in Reference 6. Further modifications of the prior art for the purpose of the present disclosure will be presented in the description of specific embodiments of the invention that follows. In addition to receiving the returned energy from the region 91, the stylet 50 also receives a returned optical energy from a sensing region 54 within the distal end 51. The sensing region 54 is characterized by its length L_{sensing} where information about strain distribution in the distal end 51 is obtained using the methods of distributed optical sensing described in subsequent sections of the present disclosure.

Disposed within the shaft 53 of the stylet, there is an optical energy guide 70 that channels the interrogating optical energy between the distal and the proximal ends of the stylet as illustrated in FIG. 2B. Examples of the optical energy guide 70 include a single-mode optical (SM) fiber, an elliptical core fiber, a polarization preserving (PM) fiber, a multimode-mode (MM) fiber, a double clad fiber or dual clad fiber (DCF) including a DCF with an eccentric core, a micro-structured or photonic crystal fiber (PCF), a multi-core fiber (MCF), fiber bundles or a plurality of separate fibers fabricated by any standard fiber-optic processes. The combination of the above optical waveguides or their splicing in one waveguide can be used. In a specific embodiment of FIG. 2B, the optical energy guide 70 is a DCF with an eccentrically positioned inner core, the eccentric DCF is further fusion spliced, at its distal end, to a focusing element 75 that aggregately consists of a section of a coreless fiber 73 and a section of a gradient index fiber 74. The focusing element 75 has also an attached directing element 76 exemplified by a right angle prism in the specific embodiment of FIG. 2B. Other embodiments of the internal imaging components 70, 75, and 76 of OCT probes that have been described before in Reference 4-6, together with interfacing arrangements in imaging processing consoles, can be used in the imaging stylet of the present invention in accordance with the methods of OCT and spectral imaging.

In some embodiments of the present inventions, as shown in FIG. 2B, the energy guide 70 with the attached focusing element 75 and the directing element 76 are structured to be fully rotatable or slideable within a lumen of the stylet body 53. In these embodiments, the internal imaging components of the stylet 50 are actuated with a flexible rotary shaft or a torque coil 65 powered by proximally located motors in the console 100 to generate spirally scanned patterns in the imaged region 91. The spiral scanning constructs, at least for some embodiment, 3D image data as illustrated in FIG. 2C. In some other embodiments, a micro-motor, or MEMS, or piezo, or electrostatic, or electromagnetic scanning actuator is disposed at the distal end of the stylet to generate scanned patterns for imaging. In yet some other embodiments, the imaging elements 70, 75, and 76 are fixed to the stylet body and imaging scanned patterns are obtained by repositioning the stylet itself within a tissue. In all embodiments, the guide 70 is structured to measure distributed strain along a portion of its length, in addition to channeling the imaging optical energy to and from the imaging region 91. The length of this strain sensing portion within the guide 70 defines the length of the sensing region 54, L_{sensing}, of the stylet distal end 51.

Turning now the attention to the system console, the console 100 of the medical apparatus of the present invention includes arrangements shown in the schematic diagram of FIG. 2D. A drive unit 110 mates with the stylet 50 to transceive the interrogating optical energy from the console to the stylet. The drive unit 110 also couples rotational or translational motion to the stylet 50 in embodiments with proximal scanning. In embodiments with distal scanning, the drive unit 110 supplies, in addition or alternatively to the mechanical motion, power or control signals to a distal scanning mechanism of the stylet 50. The operation of the drive unit 110 is controlled by a data processing and control arrangement 180 which further communicates with a user interface arrangement 190 and a data storage arrangement 170. Aggregately, the arrangements 180, 190, and 170 constitute a computer-processing unit (CPU) with a user interface in the present disclosure. The drive unit 110 also operably communicates with a strain sensing arrangement 150 and an image acquisition arrangement 140 via a multiplexing/demultiplexing arrangement 130. Further details of the drive unit 110 and its interaction with components of endoscopic OCT and spectral imaging systems have been described before in Reference 4-6 which are fully incorporated by reference in the present disclosure.

When in operable communication with the stylet 50, the strain sensing arrangements 150 performs spatially-resolved measurements of strain within the sensing length of the energy guide using the methods of distributed fiber-optic sensing. Resultant measurement data is outputted to the data processing and control arrangement 180. In some embodiments, the strain sensing arrangements 150 is structured to use the methods of Optical Fourier Domain Reflectometry (OFDR) for distributed strain sensing as described in a patent application by Foggart et al [Ref 9] which is fully incorporated by reference herein. In some other embodiments, the methods of Optical Time Domain Reflectometry (OTDR), or Optical Low Coherence Reflectometry (OLCR), or Microwave Photonics are used for distributed strain sensing [Ref 9-10]. Also, the methods of modifying optical fibers to enhance sensitivity of distributed strain sensing are in the scope of the present invention. These methods include exposing fibers to UV radiation or doping fibers to enhance Rayleigh back-scattering. The methods of enhancing strain sensitivity also include inscribing Fiber Bragg Gratings, or chirped gratings, or random gratings within the sensing length of the energy guide of the stylet 50.

The image acquisition arrangement 140 generates the interrogating optical energy, receives the tissue-encoded returned optical energy, and converts the returned optical energy into image data in accordance with the methods of OCT and spectral imaging that have been described before [Ref 4-6]. When operating, the image acquisition arrangement 140 outputs the image data to the data processing and control arrangement 180 and communicates with the multiplexing/demultiplexing arrangement 130, the arrangement 130 couples the strain sensing portion and the image portion of the interrogating or returned optical energy to or from the drive unit 110. Wavelength division multiplexing, or spatial multiplexing, or time multiplexing, or frequency domain multiplexing known in the field of fiber optics are within the scope of the present invention for use in different embodiments of the multiplexing/demultiplexing arrangement 130 as will be described in further detail in sections of this disclosure that follow.

Referring now to FIG. 2E and in further reference to FIG. 2D and FIG. 2B, an exemplary implementation of the strain sensing arrangement 150, the image acquisition arrangement 140, the multiplexing/demultiplexing arrangement 130, and the drive unit 110 are given for an embodiment that provides distributed strain sensing combined with OCT imaging and Fluorescence Lifetime Imaging (FLIM). In this specific embodiment, an OFDR source 151, which is preferably an akinetic fiber-coupled laser with tunable wavelength also referred to as a swept source, connects with a fiber optic interferometer via a fiber coupler or splitter 152, the interferometer having a reference path or reference arm 153 with a reference mirror 154 and a signal path or signal arm 155 that connects to the imaging stylet 50 via a fiber connector 112. The signal path further incorporated a fiber-coupled wavelength division multiplexer (WDM) 131, a dual core fiber coupler (DCFC) 132, and a fiber optic rotary joint (FORJ) 111, the FORT is translated and its rotor is rotated by a motorized multi-axis stage arrangement 113 to actuate proximal scanning in the imaging stylet. Accordingly, the fiber connector 112 attached to the rotor of the FORT 111 also continuously rotates during operation while a receiver 156 detects an OCT or OFDR interferogram in accordance with the methods of OCT imaging and distributed fiber-optic strain sensing. Resultant electrical signals outputted by the receiver 156 are then digitized by a digitizer or analog-to-digital converter (ADC) 157.

The specific embodiment of FIG. 2E also incorporates a plurality of spectral sources 141 that provide interrogating optical energy to excite tissue fluorescence. For example, a spectral source can be a laser diode source with a wavelength within the absorption band of an intrinsic or extrinsic tissue fluorophore, the laser diode source intensity being modulated in accordance with the methods of Fourier-domain FLIM (FD-FLIM) [Ref 11]. Alternatively, the laser diode source 141 can be a pulsed laser sources in accordance with the methods of Time-domain FLIM (TD-FLIM) [Ref 11]. In the embodiment of FIG. 2E, the spectral sources 141 are coupled to the interferometer signal arm 155 via the WDM 133 so that the optical energies outputted by the OFDR source 151 and by the spectral sources 141 are combined to propagate in a same single mode fiber (SMF), the said SMF constitutes a portion of the signal path 155 of the interferometer. The DCFC 132 then couples the propagating optical energy in the SMF to an inner core of a DCF that constitutes another portion of the interferometer signal arm 155 between the DCFC and Fiber Connector 112. The said DCF is also incorporated in a FORJ stator and the FORJ rotor so that both inner and outer cores of the DCF are rotatably optically coupled within the FORT as was described in further details before [Ref 6]. In the embodiment of FIG. 2E, as also shown in FIG. 2B, the energy guide 70 of the stylet 50 is also made of a DCF that channels the interrogating optical energy and the returned optical energy for strain sensing and OCT imaging in the DCF inner core. At the same time, the outer core of the DCF guide 70 channels fluorescence emission collected from the tissue. The collected fluorescence emission is then coupled via the FORJ 111 back to the DCFC 132 that outcouples the optical energy propagating in the DCF outer core via an MMF with an output coupler 135 towards dichroic mirrors or spectral filters 134. Different wavelength bands in the outcoupled fluorescence emission are then separated by the dichroic mirrors 134 and detected by receivers 142. The detected fluorescence signals are digitized by the digitizer 157 for digital signal processing and analysis in accordance with the methods of FD-FLIM. Alternatively, the methods of TD-FLIM are used in some embodiments of the invention in which the receivers 142 are time-correlated photon counting modules enabling time-resolved detection of fluorescence spectral bands.

In the exemplary embodiment of FIG. 2E, the OFDR source and the interferometer are advantageously used both for distributed strain sensing and for OCT imaging. Preferably, the wavelength tuning range of the akinetic swept source 151 changes for strain sensing relative to the tuning range used for OCT imaging while keeping the sampling rate of the digitizer 157 constant in this specific embodiment. By doing so, in accordance with the methods of OFDR, the maximal interrogated depth can match L_{sensing} or L_{imaging} for sensing and imaging modalities, respectively. It is clear from the description of this exemplary embodiment that alternative embodiments with separate OCT and OFDR sources or with separate interferometers for OCT imaging and OFDR strain sensing are also within the scope of the invention. Alternatively or in addition, an OFDR source for strain sensing can be simultaneously used for spectral imaging, for example to excite fluorescence of an endogenous or exogenous fluorophore. Also, the spectral domain modalities of OCT and distributed strain sensing when, for example, the OFDR source 151 is replaced with a broadband optical energy source and the receiver 156 is replaced with a spectrometer are within the scope of the invention. It is also clear from the description of the exemplary embodiment of FIG. 2E and from descriptions that follow how other spectral modalities such as, for example, two-photon FLIM, Raman, Near-infrared Reflectance Spectroscopy (NIRS) can be combined with OCT and strain sensing for the methods of the present invention.

### Position Calculations.

Now we proceed to describe how the console 100 calculates a position of the stylet in a medical procedure with the system of the invention by using a fusion, that is by using a complimentary processing, of intra-operatively acquired image and strain sensing data. In the following description we will interchangeably use three reference frames associated with 1) fixed space (inertial frame), 2) a moving distal end of a probe, and 3) patient tissue, i.e., an anatomical structure of interest within a patient body. These three reference frames can be represented with orthogonal sets of unit vectors [\hat{x}ₛ ŷₛ,\hat{z}ₛ], [\hat{x}ₚ ŷₚ,\hat{z}ₚ], [\hat{x}ₜ ŷₜ,\hat{z}ₜ] for the fixed space, the probe space, and the tissue space respectively. We choose to orient the \hat{z}ₚ along the longitudinal axis of a stylet distal end.

The following description of the fusion of image data and strain sensing data for the purpose of the present invention will be better understood by referring first to a general structure of a 3D image data set acquired by the stylet in some embodiments illustrated in FIG. 2C. In these embodiments, the stylet distal end 51 contains internal rotating imaging elements within a transparent portion of the stylet body. When the distal end 51 (or the internal rotating imaging elements) is repositioned, 3D image data is acquired from a continuous spiral surface centered on a trajectory of the distal end 51. The spiral surface is formed by rotating a side exiting beam of optical energy 92 also referred to as a A-line in the field of OCT imaging. The A-line has a characteristic imaging scan depth L_{imaging}. When the repositioning is slow, the continuous spiral surface can be conveniently approximated by a stack of plane 2D frames formed by rotating the A-line (freeze and hop approximation). Thus a 3D image data set, at least for some embodiments, can be represented by a stack of the 2D frames, or B-scans Bᵢ[n, m] further consisting of 1D A-lines Aₙ[m] of image data points, or pixels. More detailed descriptions of A-lines, which represent reconstructed depth profile of the reflected imaging energy, is given in References 4-7. Modifications of this general structure of image data for the purpose of the present disclosure will be given when describing specific embodiments of the invention.

Turning now attention to FIG. 3A, an exemplary method of reaching a tissue target 530 with the medical instrument 200 is provided using the human lungs as a specific example of an anatomical structure 500. In this navigational procedure, the endoscope 310 is advanced endoluminally to a region 505, the size of a smallest reachable lumen in the region 505 being determined by the outer diameter of the endoscope distal end. To reach a more peripheral region 510, a steerable guide sheath advances via the working channel of the endoscope 310 to extend endoluminally beyond the endoscope distal end to form an extended working channel (EWC) 350 for endoluminal deployment of endoscopic instruments. The size of a smallest reachable lumen in the region 510 is determined by the outer diameter of the EWC distal end. Finally, the target tissue 530 within an even more peripheral region 520 is reached transluminally, by piercing luminal walls with the instrument 200. During all endoluminal and transluminal advancements, the stylet 50 is positioned within the medical instrument 200 to measure distributed strain or to image lumens or surrounding tissue. Because the region 505 can be visualized and registered in the field of view (FOV) of high-quality imaging of the endoscope 310, a position of the stylet (and the medical instrument) in this region is generally known with high accuracy. However, localization of the medical instrument 200 within the anatomical structure 500 in more peripheral regions beyond the region 505 cannot be accurately determined by the endoscope. Therefore, one goal of the method of the invention is to navigate an instrument to a target while tracking a position of the instrument on its path from the region 505 to the target tissue 530 within the region 520, the position tracking relying on the image or strain sensing data acquired by the stylet as described below and as further illustrated in FIG. 3B and FIG. 3C.

Referring now to FIG. 3B, an endoluminal advancement of the medical instrument 200 in the region 510 involves an iterative estimation of a stylet position at each advancement step i, i+1, . . . i+k. The iterative position estimation uses a state model of the stylet and a measurement model that relates a position of the stylet with strain sensing data or image data acquired by the stylet. Accordingly, the system of the invention provides a positional feedback for steering a nested arrangement of the ECW 350, the instrument 200, and the stylet 50 within a branching luminal tree towards a target branch.

Referring now to FIB 3C, advancing the instrument further transluminally and placing it within the target tissue 530 located in the region 520 involves extending a nested arrangement of the medical instrument 200 and the stylet 50 beyond the ECW 350 while continuing the iterative estimation of a stylet position at each advancement step i′, i′+1, . . . i′+k′ using the state model and the measurement model of the stylet. Accordingly, the system of the invention provides positional feedback for aligning the nested arrangement of the instrument 200, and the stylet 50 towards a target before puncturing a luminal wall, for confirmation of wall puncturing, or for confirmation of reaching the tissue target 530.

Proceeding now to a detailed description of the iterative position estimation of the stylet of the invention, FIG. 3D shows an exemplary recursive data acquisition- and position calculation sequence 700 that uses a previous state of the stylet, i.e. a position and a pose of the distal end of the stylet determined at an i−1 iteration, to estimate a current i-th state of the stylet using a current strain sensing data acquired by the stylet in an acquisition sub-process 720 or a current image data acquired by the stylet in an acquisition sub-process 730. The algorithm 700 is based on Extended or Unscented Kalman Filter [Ref 12] or, more generally, on any Bayesian Filter that first predicts a new state in a prediction sub-processes 710 using a stylet state model without invoking current sensing or image data. In accordance with the methods of Bayesian filters, the said state model, also referred to as a process model in this disclosure, is defined as a conditional probability of a current stylet state given a previous stylet state. Accordingly, the prediction sub-process 710 provides a prior estimate of the stylet current state. The algorithm 700 also uses a measurement model, also called an observation model in this disclosure, defined as a likelihood of observing a strain distribution in the sensing region or observing an image data in the imaging region for a given stylet state. In accordance with the methods of Bayesian filters, the measurement model corrects the state prediction in an estimation sub-process 770 thus providing a posterior estimate of the current state. An exemplary state model that can be used for the filter process 700 is given by:

\vec{X}ᵢ=(\vec{r}ᵢ,\hat{x}_{p,i},ŷ_{p,i},\hat{z}_{p,i})

\vec{r}ᵢ=\vec{r}_{CL}(sᵢ,nᵢ,mᵢ)+δ\vec{r}ᵢ

\hat{ξ}_{p,i}= [symbol] _{z}(θ_{z,i}) [symbol] _{y}(θ_{y,i}) [symbol] _{z}(θ_{z,i})\hat{ξ}_{CL,i}(\vec{r}_{CL}(sᵢ,nᵢ,mᵢ));ξ=x,y,z

Here i denotes an iteration step, \vec{X}ᵢ is a state vector with a position \vec{r}ᵢ and a pose [\hat{x}ₚ, ŷₚ, \hat{z}ₚ], respectively. The stylet position is further decomposed into a position of its distal end on a branching tree of lumen centerlines \vec{r}_{CL}(sᵢ nᵢ, mᵢ) and a deviation δ\vec{r}ᵢ of the distal end from the centerline tree, where sᵢ is a distance along the centerlines from an origin of the centerline tree, nᵢ is an index identifying a proximal brunching point common for current branches, and mᵢ is an index identifying a current branch. The branching tree of centerlines \vec{r}_{CL}(sᵢ, nᵢ, mᵢ) is pre-computed from the pre-operative data using the image-processing methods known in the field of medical device navigation. Further, θ_{ξ,i} denotes a rotation of the stylet reference frame during an i-th iteration around a principal axis of a moving reference frame [\hat{x}_{CL}, ŷ_{CL}, \hat{z}_{CL}] defined by tangential angles to the centerlines, with [symbol] _{ξ}(θ) denoting a rotation matrix for a rotation with an angle θ around an axis \hat{ξ}ᵢ. The exemplary state model for the filter process 700 is further given by:

$$P ( {\vec{X}}_{i} \vert {\vec{X}}_{i - 1} ) = {\sum }_{{m}_{i}} P ( {n}_{i} , {m}_{i} \vert {\vec{X}}_{i - 1} ) P ( {s}_{i} , δ {r}_{ξ , i} , {θ}_{ξ , i} \vert {\tilde{X}}_{i - 1} ) ; ( ξ = x , y , z )$$

$${s}_{i} = {s}_{i - 1} + {τ}_{i}$$

$$δ {\vec{r}}_{i} = δ {\vec{r}}_{i - 1} + {\vec{t}}_{i}$$

$${\vec{t}}_{i + 1} = {\vec{t}}_{i - 1}$$

$${τ}_{i} = {τ}_{i - 1}$$

$${θ}_{ξ , i} = {θ}_{ξ , i - 1} ; ( ξ = x , y , z )$$

$${n}_{i} = \{ \begin{matrix}n ( {m}_{i - 1}^{max} ) , if {s}_{i - 1} ∈ Δ {s}_{branch} ( {n}_{i - 1} ) \\ {n}_{i - 1} , otherwise\end{matrix}$$

$$P ( {n}_{i} , {m}_{i} ) = \{ \begin{matrix}{P}_{0} ( {m}_{i} ) , if {s}_{i - 1} ∈ Δ {s}_{branch} ( {n}_{i - 1} ) \\ P ( {n}_{i - 1} , {m}_{i - 1} \vert {\vec{X}}_{i - 1} )\end{matrix}$$

Here P(\vec{X}ᵢ|\vec{X}ᵢ₋₁) is a conditional probability of the current state vector with P(nᵢ, mᵢ|\vec{X}ᵢ₋₁), and P(sᵢ,δr_{ξ,i}, θ_{ξ,i}|\vec{X}ᵢ₋₁) denoting conditional probabilities of current integer coordinates (indexes of branching points and current brunches) and current continuous coordinates of the state vector, respectively. The conditional probability P(sᵢ, δr_{ξ,i}, θ_{ξ,i}|\vec{X}ᵢ₋₁) includes all positional and angular accelerations and other noise factors known as a process noise in the field of Kalman filtering, the process noise terms being omitted here for brevity. In other words, updates in the Kalman process of the exemplary state model at each iteration step are described by transitions τ of the distal end of the stylet along the centerlines of the lumen tree, additional translation vectors of the distal end \vec{t}=[tₓ, t_{y}, t_{Z}]^{T}, and the rotational angles θ_{ξ,i}. Furthermore, the exemplary state model assumes that the probability of the stylet to be located within a branch mᵢ does not change unless the distance sᵢ along the centerline is within a predetermined range Δs_{branch}(nᵢ₋₁) from a previous branching point nᵢ₋₁. When the model distance sᵢ₋₁ reaches the range Δs_{branch}(nᵢ₋₁), the identifier of the current branching point in the model switches to a distal branching point associated with a branch of a maximal probability determined at at the previous iteration step mᵢ₋₁^{max}. Reaching the range Δs_{branch}(nᵢ₋₁) also resets the conditional probability of the stylet to be located within a branch mᵢ to a predetermined prior probability P₀(mᵢ).

Referring again to FIG. 3D, an exemplary measurement model includes a likelihood of observing a strain distribution in the stylet sensing region assuming, at each iteration of the process 700, that the stylet shape adapts to the branching centerline tree with the stylet distal end located at \vec{r}_{CL}(sᵢ, nᵢ, mᵢ). In accordance with the methods of Kalman or Bayesian, the exemplary measurement model provides, at each iteration step, a correction of predicted values of sᵢ, nᵢ, mᵢ in a calculation sub-process 760 using an observed strain distribution calculated in a calculation sub-process 750. The exemplary measurement model also includes a likelihood of observed image data for a given stylet state; the said image data include Doppler shifts or speckle correlations, or image similarities, or position identifiers as was described in Reference 7 which is incorporated herein by reference in its entirety. Accordingly, the calculation sub-process 760 uses Doppler shifts between B-scans calculated in a sub-process 751, or speckle correlation analysis of B-scans calculated in a sub-process 751, or a similarity between the image data and the pre-operative data calculated in a sub-process 753, or position identifiers calculated in a sub-process 754. Corrections to the predicated state are then used jointly to update the stylet state in the estimation sub-process 770 in accordance with the methods of Kalman or Bayesian filtering.

From the description and the references provided, it is clear how the Kalman filter process can be modified to include degrees of freedom of a medical tool in a modified state model or a measurement model. In accordance with the methods of Kalman and Bayesian filtering, a position and a pose of the tool can be estimated by adding independent tool state variables to a process model. Examples of such independent variables are a variable protrusion of a needle or its angular orientation relative to the stylet. An exemplary modification of the measurement model relates Doppler shifts from a tool region in image data set with the tool state components using a known location of a tool portion in the stylet imaging region. An exemplary modification of the process model includes a known spatial relationship between the visualized portion of the tool and a portion of the tool that reaches a tissue target. This method of analyzing tool region in image data is not limited to Doppler shifts but can be also applied to other measurement models, as long as a process model incorporates a known spatial relationship between an imaged portion and a working portion of a medical instrument.

Recursive Kalman or Bayesian filters described in relation to FIGS. 3A-3D allow complementary fusion of different methods of using strain data and image data for estimation of a stylet position. These different methods can be also referred to as different data channels in a measurement models to emphasize different processing steps and different physical mechanisms underlying stylet state calculations. These different channels are also known as different sensors in the field of Kalman filters [Ref 12]. Inaccuracies associated with different data channels might have different spatial scales. As a result, a hybrid estimation with sensor fusion or fusion of different channels of strain and image data improves accuracy and robustness of determining a stylet state. Also, a degree of freedom in a state model can be estimated more accurately with a specific modality in image data, based on prior assumptions of the state model (empirical confidence factors) or intra-procedural estimation of measurement model noises in corresponding channel of image data.

Processes of estimating a stylet state described so far treated tissue motion as a noise term in a process model. Alternatively or in addition, tissue deformation maps can be independently estimated and included in a process of calculating the stylet position to a target as was described before in Reference 7. For example, Doppler shifts between sub-regions of a B-scan can be analyzed to generate tissue deformation flow, and then, by integrating, a tissue deformation map within the B-scan. In case of speckle correlation analysis, translation vectors that maximize correlations of each block of image data correspond to tissue deformation vectors, once an average translation vector is subtracted. Similarly, a non-rigid analysis of similarity of image data can be used to determine deformation maps that can be then included in a process model and a measurement model. A non-rigid analysis, in a context of this application, means an image data similarity analysis of sub-sets of image data sufficiently small to be considered rigid, with independent transforms of the rigid sub-sets of image data at each iteration step. Overall tissue motion relative to the stylet can be independently estimated with image data acquired while strain data is used to track position and orientation of a probe relative to the fixed space. A pre-determined model of tissue deformations, in particular tissue deformations caused by an interaction with the medical tool can be used in a Kalman filter model, for example when a tissue is dragged by a needle traversing the tissue during transluminal or interstitial placements. Overall tissue motion (i.e. relative motion of rigid tissue frame, without deformation, with respect to the space reference frame) can be also included explicitly in the model or can be estimated with external position sensors.

### First Preferred Embodiment

In references to FIGS. 4A-4D and also to FIG. 2B, we now proceed to a detailed description of a first preferred embodiment of the system of the invention in which the stylet incorporates a sensing optical energy guide structured to be fully rotatable inside a lumen of the stylet body. FIG. 4A shows a cross-section of the stylet 50 within the strain sensing region 53 for this embodiment, showing also exemplary dimensions of its key structural elements. Here the rotatable optical energy guide of the stylet is the eccentric DCF 70 described before in reference to FIG. 2B. Referring back to FIG. 4A, The DCF 70 is co-axially attached to the previously described torque coil 65 that rotates during imaging co-axially and within a sealed outer tube that constitutes the stylet body in the distal end 51 for this embodiment. The inner, single mode, core of the DCF is structured with a radial offset with respect to the DCF axis. Consequently, there is a radial offset between the inner core and a rotation axis of the DCF 70 and the torque coil 65 for the arrangement of FIG. 4A. Also, there is a radial offset between the inner core of the rotatable DCF 70 and a neutral bending axis of the stylet body. As a result of the eccentric offsets, the strain distribution S within the inner single mode core of the DCF in the strain sensing region becomes a time-varying function S(t,l) when the optical energy guide 70 rotates. Here t denotes time of strain measurement and 1 denotes position coordinate along the stylet length. FIG. 4D further illustrates temporal and spatial distribution of strain in the strain sensing region of the stylet during operation. Here three time-varying strain functions that oscillate with a period of the rotation at three different exemplary locations lₘ, lₙ, and lₚ are shown. Amplitudes A(1) and phases θ(1) of the strain oscillations are affected by shapes that the stylet takes while navigating to a target. In other words, the time-varying strain distribution S(t,l) correlates with a position of the stylet. Accordingly, the said amplitude and phases of strain oscillations can be incorporated in the measurement mode of the estimation process 700 in accordance with the method of the present invention.

Referring now to FIG. 4B, we describe details of the proximal end 52 of the stylet and also details of the FORJ 111 and the fiber connector 112 for the first preferred embodiment. FIG. 4B shows, cross-sectionally, a ferule 55 that holds the eccentric DCF 70 of the stylet in the proximal end. The ferrule 55 has an eccentric bore to align the inner core of the DCF 70 with an inner core of a rotationally symmetric DCF 77 that guides optical energy in the rotor portion of the FORJ 111. The rotationally symmetric DCF 77 is secured in the fiber connector 112 with a standard rotationally symmetric fiber ferrule. The rotationally symmetric DCF 77 is also structured to have a diameter of its outer core to accept optical energy propagating in the outer core of the eccentric DCF 70 when the said DCFs are mated in the fiber coupler 112. The DCF 77 is then optically coupled with the same DCF that guides optical energy in the stator portion of the FORJ as was described before.

Referring now to FIG. 4C, alternatively or in addition, the first preferred embodiment can have a standard SMF 71 disposed eccentrically with respect to the rotational axis of the torque coil. In an exemplary arrangement of FIG. 4C, the eccentric placement of the SMF is accomplished by means of a MMF 72 disposed within an inner diameter of the torque coil 65 that displaces the SMF away from the rotational axis. The MMF 72 also guides optical energy for spectral imaging. For the exemplary arrangement of FIG. 4C, the proximal end of the stylet, the mating fiber connector of the FORJ, and the FORJ itself are modified to include standard duplex fiber couplers and a standard dual channel FORJ with one SMF channel and one MMF channel.

### Second Preferred Embodiment

Proceeding now to describe a second preferred embodiment of the invention, FIG. 5A shows the distal end 51 of the stylet protruding from the instrument 200 in this embodiment. Here the stylet emits a plurality 94 of side-exiting beams of optical energy that are fixed with respect to the stylet body. Accordingly, scanned patterns for distal imaging are obtained by repositioning the stylet itself. The said repositioning is actuated by operator manipulating the proximal end of the stylet, for example by manual pulling. Alternatively or in addition, motors of the drive unit can actuate the stylet reposition. In accordance with methods of the invention, the scanned patterns or, equivalently, positions of the side-exiting beams 94 are tracked with the process 700 of FIG. 3D during the repositioning. This positional information is used to combine and remap one dimensional image data acquired from each side exiting beam into 2D or 3D image data with extended imaging range. Referring now to FIG. 5B and FIG. 5D that show cross-sections of the stylet perpendicular to the stylet axis and containing the said axis, respectively, the stylet body in this embodiment is a grooved wire 55, for example a grooved nitinol wire, inserted into a protective outer tube 56, for example into an FEP tube fixed to the wire with an adhesive. The said eccentric grooves accommodate attached DCFs 78 that are, therefore, also located eccentrically with respect to the neutral bending axis of the stylet. The DCFs 78 exemplify the sensing optical energy guides of the invention in this embodiment. At the distal end, the DCFs 78 are optically coupled with the focusing elements 75 and the energy directing elements 76 disposed within the grooves to form the side exiting beams of optical energy 94 as shown in FIG. 5D.

Shown in FIG. 5C and FIG. 5E is an alternative implementation of the stylet in the second preferred embodiment that includes an MCF 79, for example an MCF with six eccentric single-mode cores. The MCF 79 is fixedly enclosed in a flexible tube 58, for example in a nitinol tube. FIG. 5E also shows a single focusing element 74, for example a GRIN lens, attached to the MCF 79 to focus the optical energy exiting from the eccentric cores of the DCF and to project the optical energy towards a curved mirror 80. The curved mirror 80 directs the optical energy towards a tissue through exit windows in the tube 58 forming the plurality 94 of the side exiting beams. In this arrangement, a multi-faceted reflective prism can be used as a directing element instead of the curved mirror 80. In one alternative arrangement for the second preferred embodiment shown FIG. 5F, a wide-angle lens 81, for example a diffractive lens or a metalens, is attached to the focusing element 74 to direct optical energy to a tissue to form the side exiting beams 94.

Because there is no distally rotating energy guides in this embodiment, the drive unit of the console can be advantageously simplified to use fan-out arrangements of fiber connectors known in the field of fiber optic spatial multiplexing instead of using FORJs. Also, it is clear from the description and the references provided, that strain distributions measured in the strain sensing regions of single-mode cores of the individual DCFs as illustrated in FIG. 5D or in separate single-mode cores of the single MCF as illustrated in FIG. 5E can be advantageously combined or fused using Kalman or Bayesian filtering to improve accuracy of navigation and position tracking in accordance with the methods of the present invention.

### Third Preferred Embodiment

In a third preferred embodiment, the system of the invention is structured to generate scanned patterns for imaging using distal scanning. Referring first to cross-sectional views presented in FIG. 6A and FIG. 6C, the stylet in this embodiment includes an MCF 82 attached to an XY actuator 66, for example to a segmented piezo-tube actuator, the said XY actuator is disposed distally within the tube 58 of the stylet body. In a specific example illustrated in FIG. 6A and FIG. 6C, the MCF 82 has one center single-mode core, three eccentric single-mode cores, and a common multi-mode core enclosing the said four single-mode cores. The MCF 82 exemplifies the sensing optical energy guide of the invention that uses, in accordance with methods of the present invention, the center single-mode core for tissue imaging, the three eccentric single-mode cores for strain sensing, and the common multi-mode core for collecting optical energy returned by tissue for spectral imaging. Referring now solely to FIG. 6C, the distal end of the DCF 82 has a stepped outer diameter formed by fusing a section of an SMF 83 to couple optical energy between the center single-mode core of the MCF 82 and the SMF 83. In operation, the XY actuator 66 scans the tip of SMF 83 laterally to form a circular scanning pattern projecting optical energy to the focusing element 75 and then to the directing element 80. The element 80 directs the optical energy further towards a tissue via windows in the stylet body forming a side-exiting beam 92 that continuously rotates during imaging to generate a B-scan. An exemplary implementation of the XY actuator 66 with associated methods and structures based on using segmented piezo tubes has been described before in Reference 8 which is incorporated by reference herein in its entirety. Also, the exemplary arrangement of FIG. 6C shows a hollow reflective cylindrical optical element 84, for example a metal coated glass capillary, disposed within the stylet tube 58 and located between the MCF 82 and the focusing element 75. The tube element 84 concentrates returned optical energy from a tissue onto the multi-mode core of the MCF 82 thus improving collection efficiency of the stylet for spectral imaging.

Alternative example of the third preferred embodiment uses torsional distal scanning. Referring first to cross-sectional views of FIG. 6B and FIG. 6D, a torsional actuator 67 is attached distally to the outer tube 58 of the stylet. The said torsional actuator holds the MCF 79 having the eccentric cores as described before. Optically coupled to the distal end of the MCF 79 are the previously described focusing element 75 and the wide-angle diffractive directing element 81 that form an arrangement outputting the plurality of the side-exiting beams 94. In the specific example of the FIG. 6D, the plurality 94 is directed towards a tissue via a transparent distal tube 61, for example via a polyimide tube attached distally to the outer tube 58 and sealed with the cap 57. Also, in this specific example the focusing element 75 and the directing element 81 are attached to the distal end of MCF 79. In alternative arrangements, the focusing element or the directing element can be attached to the outer body or the cap of the stylet thus decoupling mechanically from the MCF 79. Also, a curved or faceted mirror can be used as a directing element in alternative arrangements. During imaging, the torsional actuator 67 reciprocally rotates the distal end of the MCF 79 and, correspondingly, the plurality of beams 94 around the axis of the stylet, preferably with an amplitude of the reciprocal rotation ϕ(t) exceeding the angular separation of the side-exiting beams as shown in FIG. 6E. Referring again to FIG. 6E, an exemplary distal end structure with the torsional scanner 67 has a polyimide tube 62 holding the DCF 79 with the attached focusing element 75 and the directing element 61. The tube 62 is further attached to the outer body 58 (not shown in FIG. 6E) of the stylet while the distal end of the MCF 79 is free to rotate within the tube 58. Electroplated copper coils are deposited distally on the outer surface of the polyimide tube and covered with isolating material, for example with an additional layer of polyimide tubing, the said copper coils are connected proximally with the drive unit of the console. The distal tip of the DCF 79 is also coated with a magnetic material magnetized to have a permanent magnetization vector M oriented at an angle with respect to the axis of the MCF as shown in FIG. 6E. During imaging, the drive unit of the console supplies an alternating electrical current to the coils 63 by to generate a time-varying magnetic field H(t) at the distal end of the stylet. Accordingly, the said magnetic field interacts with the magnetization vector M of the MCF 79 causing reciprocal oscillation of the MCF tip around its axis as a result. Accordingly, the plurality of beams 64 is oscillating around the stylet axis to construct image data while positions of the beams together with the overall position of the distal end of the stylet are tracked in accordance with the methods of the invention using the process 700 of FIG. 3D.

### Exemplary Medical Procedures

Proceeding to explain further the method and associated medical instruments of the invention, details of an exemplary medical procedure of a non-surgical bronchoscopic biopsy of lung nodules found by chest CT are provided. This procedure is presented in reference to FIGS. 7A-7F and also to FIGS. 3A-3C and in further reference to a detailed description of image-guided interventions given before in Reference 7. Referring first to FIG. 3A, the target tissue 530 is a suspicion indeterminate lung nodule that needs to be biopsied bronchoscopically. First, a practitioner (an interventional pulmonologist) determines, in a planning step preceding intervention steps, a pathway to the nodule (also called a lesion) in the patient airway tree using a software that segments airways in the CT data; the said pathway includes pre-computed airway centerlines that will be used for subsequent position tracking in accordance with the method of the present invention. During the planning step, the practitioner also determines an appropriately-sized set of a guide sheath, the stylet of the invention, and biopsy tools to be used during the bronchoscopic procedure, for example selecting the ECW 350, the flexible transbronchial needle 200, and the stylet 50 with outer dimensions indicated in FIG. 7A. Referring again to FIG. 3A, an intervention portion of the biopsy procedure begins with brining the bronchoscope to the region 505 via the pre-determined pathway. Once the bronchoscope cannot advance any further, the imaging stylet 50, the needle 200 and ECW 50 are navigated to more peripheral airways in the region 510 via a working channel of the bronchoscope. In this navigation part of the biopsy procedure, the needle is advanced towards the target using position tracking and imaging guidance of the stylet described above and using steering of the ECW. Also, as shown in FIG. 7A, in the navigation portion the stylet, a tip of the needle is withdrawn within a hub of the needle, the needle and the stylet are located within the ECW, and the stylet images surrounding tissue via a transparent tip of the ECW. Accordingly, during this portion of the biopsy procedure, the stylet, the needle, and the ECW are advanced and aimed towards the target together, in a nested navigation arrangement shown in FIG. 7A. Once the said navigation arrangement reaches proximity to the lesion and is aimed towards the lesion, the needle extends from the tip, puncturing the airway wall for an interstitial needle placement in the target. During this puncture step, the stylet advances together with the needle tracking the needle position with strain sensing and interstitial imaging of parenchyma in accordance with the methods of the invention. Then, the needle is pulled back and the stylet acquires image data to confirm placement in the target or to determine if the needle needs to be repositioned. If the target placement is confirmed, stylet is withdrawn and the practitioner advances the needle to collect tissue biopsy samples. The image data acquired in the confirmation portion of the procedure is also stored to be passed to a pathologist to assist histological analysis of biopsy tissue samples.

Referring now to FIG. 7B and FIG. 7C, we describe examples of medical biopsy instruments of the present invention that collect biopsy samples in sideway direction. FIG. 7B shows a 3D view of a distal end of a side-cutting biopsy device 230 having a single-lumen flexible shaft 231 that extends to a proximal end of the instrument. The shaft 231 is sealed at its distal end with a rigid tip 232 bonded to the shaft with an adhesive or a thermal fusion process. The rigid tip 232 has a side window 233 for tissue cutting, and a through hole 234 for stylet accommodation. In some embodiments, the biopsy device 230 has also a transparent tube placed inside the internal lumen of the flexible shaft 231 and sealingly secured to the through hole 234. Thus the said transparent tube forms a dedicated lumen for sealing the stylet from the remaining lumen used as a tissue channel for specimen collection. Furthermore, at least in some embodiment, specimen collection is facilitated with vacuum provided by a proximal vacuum port of the device connected to the internal cavity of the rigid tip 232 via the tissue channel of the shaft 221. During biopsy, the device distal end is aligned with respect to a target by pushing, puling or rotating (torqueing) the device distal end with a user inputs at the proximal end, using image and position guidance provided by the stylet 50.

In some embodiments, the side cutting instrument 230 has also a moveable internal cutting element extended inside the internal lumen of the flexible shaft 231 to its proximal end. The said cutting element is repositioned by a handle connected to the cutting element at its proximal end. When a tissue is prolapsed inside the window 233, the practitioner pulls the cutting element while the stylet 50 images the harvested tissue providing biopsy feedback in real time. Further details of side-collecting biopsy instruments with moveable internal cutting elements that can be used with the system of the present invention are provided in Reference 7.

FIG. 7C shows a 3D view of a distal end of another side-collecting biopsy device 240 that can be used in the system of the present invention. The instrument 240 has a flexible multi-lumen extrusion 241, with one internal lumen accommodating the stylet 50 and the other internal lumens connected to cutting windows 243 machined in a rigid tip 242. Each cutting window is connected to its own source of vacuum via a dedicated tissue channel to anchor itself to tissue when vacuum is applied and to collect tissue samples independently. This multi-window arrangement minimizes a need to torque the distal end of the instrument 240 during alignment to a target thus improving alignment capabilities of this side-cutting biopsy device.

Proceeding now to FIGS. 7D and 7E, we present examples of biopsy instruments of the invention with improved tissue collection from forward direction. FIG. 7D shows a distal end of a core biopsy needle device 250 having a multi-lumen needle with a lumen 252 accepting tissue samples and a lumen 251 accommodating the stylet 50. Additionally, to facilitate imaging of surrounding tissue during device placement confirmation within a target, the needle 250 has an imaging slot 253 as shown in FIG. 7E.

FIG. 7F illustrates a forward-collecting biopsy device 260 with a multi-lumen coring needle 261 and the imaging stylet 50 deployable with a lateral offset with respect to a longitudinal axis of the needle 261. Such off-angle placement of the stylet is achieved by using a slideable pre-bended element 265, for example a pre-bended nitinol tube, that guides the stylet laterally when the element 256 is extended from the needle 261. The arrangement of FIG. 7F further includes a flexible outer sheath 264 with a rigid needle hub 263. A keying element attached to the needle 261 engages corresponding internal keying features in the hub 263 to lock an angular orientation of a lateral off-angle placement of the stylet 50. The distal arrangement of FIG. 7F is further structured to change an angular orientation of a lateral placement by axially repositioning the keying element away from the corresponding keying features in the hub, rotating the needle, and then returning the needle to engage the keying features in a different angular orientation. While FIG. 7F shows a multi-lumen needle, with one lumen dedicated to tissue collection, a single lumen needle can be also used by repositioning the imaging probe inside the needle lumen after a confirmation of a correct placement is obtained. Other arrangements with a lateral deployment of the imaging stylet or a lateral deployment of a biopsy needle described in Reference 7 are within the scope of the present invention. Also, energy-depositing instruments guided by the imaging stylet as described in Reference 7 are within the scope of the present invention.

### Other Considerations

It is to be understood that no single drawing used in describing embodiments of the invention is intended to support a complete description of all features of the invention. In other words, a given drawing is generally descriptive of only some, and generally not all, features of the invention. A given drawing and an associated portion of the disclosure containing a description referencing such drawing do not, generally, contain all elements of a particular view or all features that can be presented is this view, for purposes of simplifying the given drawing and discussion, and to direct the discussion to particular elements that are featured in this drawing. A skilled artisan will recognize that the invention may possibly be practiced without one or more of the specific features, elements, components, structures, details, or characteristics, or with the use of other methods, components, materials, and so forth. Therefore, although a particular detail of an embodiment of the invention may not be necessarily shown in each and every drawing describing such embodiment, the presence of this detail in the drawing may be implied unless the context of the description requires otherwise. In other instances, well known structures, details, materials, or operations may be not shown in a given drawing or described in detail to avoid obscuring aspects of an embodiment of the invention that are being discussed. Furthermore, the described single features, structures, or characteristics of the invention may be combined in any suitable manner in one or more further embodiments.

It is also to be understood that although image or strain distribution data processing steps that estimate a stylet position in this invention are explained in terms of Kalman or Bayesian filters, other data processing algorithms known in the art of position tracking are within the scope of the invention as long as these processing algorithms process data sets described in the invention.

References throughout this specification have been made to “one embodiment,” “an embodiment,” “a related embodiment,” or similar language. Such references mean that a particular feature, structure, or characteristic described in connection with the referred to “embodiment” is included in at least one embodiment of the present invention. Thus, appearances of the phrases “in one embodiment”, “in an embodiment”, and similar language throughout this specification may, but do not necessarily, all refer to the same implementation of the inventive concept. Furthermore, the phrases “in another embodiment” and “in some other embodiments” as used herein do not necessarily refer to a different embodiment, although it may. Thus as described below, various embodiments of the invention may be readily combined, without departing from the scope or spirit of the invention It is to be understood that no portion of disclosure, taken on its own and in possible connection with a figure, is intended to provide a complete description of all features of the invention.

The invention as recited in claims appended to this disclosure is intended to be assessed in light of the disclosure as a whole, including features disclosed in prior art to which reference is made.

At least some elements of a device of the invention can be controlled, in operation with a processor governed by instructions stored in a memory such as to enable desired operation of these elements and/or system or effectuate the flow of the process of the invention. The memory may be random access memory (RAM), read-only memory (ROM), flash memory or any other memory, or combination thereof, suitable for storing control software or other instructions and data. Those skilled in the art should also readily appreciate that instructions or programs defining the functions of the present invention may be delivered to a processor in many forms, including, but not limited to, information permanently stored on non-writable storage media (e.g. read-only memory devices within a computer, such as ROM, or devices readable by a computer I/O attachment, such as CD-ROM or DVD disks), information alterably stored on writable storage media (e.g. floppy disks, removable flash memory and hard drives) or information conveyed to a computer through communication media, including wired or wireless computer networks. In addition, while the invention may be embodied in software, the functions necessary to implement the invention may optionally or alternatively be embodied in part or in whole using firmware and/or hardware components, such as combinatorial logic, Application Specific Integrated Circuits (ASICs), Field-Programmable Gate Arrays (FPGAs) or other hardware or some combination of hardware, software and/or firmware components.

While the invention is described through the above-described exemplary embodiments, it will be understood by those of ordinary skill in the art that modifications to, and variations of, the illustrated embodiments may be made without departing from the disclosed inventive concepts. Furthermore, disclosed aspects, or portions of these aspects, may be combined in ways not listed above. Accordingly, the invention should not be viewed as being limited to the disclosed embodiment(s).

### REFERENCES

Ref 1: Herth M D, Felix J. F., Krasnik M D, Mark, Yasufuku M D, Kazuhiro, Rintoul M D, Robert, and Ernst M D, Armin. “Endobronchial Ultrasound-guided Transbronchial Needle Aspiration.” J Bronchol. Vol 13, Number 2; April 2006.
Ref 2: Zahid Yaqoob et al, “Methods and application areas of endoscopic optical coherence tomography”, Journal of Biomedical Optics 116, 063001 November/December 2006.
Ref 3: Matthias Baumhauer, Marco Feuerstein, Hans-Peter Meinzer, and J. Rassweiler. Journal of Endourology. April 2008, 22(4): 751-766.
Ref 4: Andrei Vertikov, U.S. patent application Ser. No. 13/832,868.
Ref 5: Andrei Vertikov, U.S. patent application Ser. No. 14/040,084.
Ref 6: Andrei Vertikov, U.S. patent application Ser. No. 15/237,517.
Ref 7: Andrei Vertikov, U.S. patent application Ser. No. 16/300,475.
Ref 8: Seibel et al, U.S. patent application Ser. No. 12/281,251.
Ref 9: Frogatt et al, U.S. patent application Ser. No. 12/047,056.
Ref 10: Tossy et al, “Fiber optic sensors for sub-centimeter spatially resolved measurements: Review and biomedical applications”, Optical Fiber Technology 43(2018) 6-19.
Ref 11: Laura Marcu, Paul M. W. French, Daniel S. Elson,” Fluorescence Lifetime Spectroscopy and Imaging: Principles and Applications in Biomedical Diagnostics” CRC Press, Jul. 17, 2014.
Ref 12: Bar-Shalom and Xiao-Rong Li, “Estimation and Tracking: Principles, Techniques and Software”, Artech House Boston, 1993; Jazwinski, A. H. “Stochastic Processes and Filtering Theory”, New York, Academic Press, 1970; Bozic, S M, “Digital and Kalman Filtering”, Edward Arnold, London 1979; Steven M Kay, “Fundamentals of Statistical Signal Processing. Estimation Theory”, Prestine Hall, N.J., 1993.

What is claimed is:

1. An image-guided system comprising:
A first arrangement configured to image a tissue, the first arrangement including:
an elongated flexible body having a proximal end and an opposite distal end, and having an internal lumen extending from the proximal end to the distal end,
an optical guide extended inside the flexible body and configured to deliver an optical energy between the proximal end and the distal end, and also configured to return a portion of the optical energy from a sensing portion of the optical guide,
wherein the optical guide is also configured to be continuously rotatable inside the internal lumen of the first arrangement around a rotation axis; and
at least one optical directing element disposed within the distal end of the first arrangement and configured to transmit the optical energy delivered by the optical guide to the tissue;
a system console including a data-processing unit with memory; the system console being in operable communication with the optical guide and configured to:
process the optical energy acquired from the tissue by the first arrangement and delivered by the optical guide to generate image data,
process the optical energy returned by the sensing portion of the optical guide to measure strain distribution data within the sensing portion when the optical guide continuously rotates inside the internal lumen of the first arrangement, and
calculate a position of the distal end of the first arrangement relative to a target in the tissue using the strain distribution data and a reference image data of the tissue; the reference image data pre-acquired and stored in data-processing memory of the console.

2. An image-guided system according to claim 1, wherein the position calculation further uses the image data acquired by the first arrangement.

3. An image-guided system according to claim 1, wherein the optical guide is disposed with a lateral offset with respect to the rotation axis.

4. An image-guided system according to claim 1, wherein the optical guide is located in an eccentric bore of a ferrule disposed in the proximal end of the first arrangement.

5. An image-guided system according to claim 1, further comprising:
a second arrangement configured to accept the first arrangement; and
the system console further configured to:
calculate a position of one of the distal end of the first arrangement and a distal end of the second arrangement relative to a target in the tissue using the strain distribution data and the reference image data of the tissue.

6. An image-guide system according to claim 5, wherein the position calculation further uses the image data acquired by the first arrangement.

7. An image-guided system comprising:
A first arrangement configured to image a tissue, the first arrangement including:
an elongated flexible body having a proximal end and an opposite distal end, and having a longitudinal axis extending from the proximal end to the distal end,
a plurality of optical guides extended inside the flexible body and configured to deliver optical energy between the proximal end and the distal end, at least some of the optical guides also configured to return portions of the optical energy from sensing portions of the said optical guides,
wherein the optical guides are immovable affixed to the flexible body and at least some of the optical guides are positioned with lateral offsets with respect to the longitudinal axis; and
at least one optical directing element disposed within the distal end of the first arrangement and configured to transmit the optical energy delivered by the optical guides to the tissue;
a system console including a data-processing unit with memory; the system console being in operable communication with the plurality of the optical guides and configured to:
process the optical energy acquired from the tissue by the first arrangement and delivered by the optical guides to generate image data,
process the optical energy returned by the sensing portions of the optical guides to measure strain distribution data within the sensing portions, and
calculate a position of the distal end of the first arrangement relative to a target in the tissue using the strain distribution data, the image data, and a reference image data of the tissue; the reference image data pre-acquired and stored in data-processing memory of the console.

8. An image-guided system according to claim 7 further comprising a common optical directing element disposed within the distal end of the first arrangement and configured to transmit the optical energy delivered by a plurality of the optical guides to the tissue.

9. An image-guided system according to claim 8 wherein the common optical directing element is a wide-angle lens arrangement.

10. An image-guided system according to claim 8 wherein the common optical directing element is a diffractive metalens.

11. An image-guided system according to claim 8 wherein the common optical directing element is a curved mirror.

12. An image-guided system according to claim 7 wherein the optical guides are dual clad optical fibers.

13. An image-guided system according to claim 7, further comprising:
a second arrangement configured to accept the first arrangement; and
the system console further configured to:
calculate a position of one of the distal end of the first arrangement and a distal end of the second arrangement relative to a target in the tissue using the strain distribution data, the image data, and the reference image data of the tissue.

14. An image-guided system according to claim 7 wherein the system console is also configured to combine image data sets from the individual optical guides, remapping the said image data using the calculated position to render a joint image of imaged tissue.

15. An image-guided system comprising:
A first arrangement configured to image a tissue, the first arrangement including:
an elongated flexible body having a proximal end and an opposite distal end, and having a longitudinal axis extending from the proximal end to the distal end,
a plurality of optical guides extended inside the flexible body and configured to deliver optical energy between the proximal end and the distal end, at least some of the optical guides also configured to return portions of the optical energy from sensing portions of the said optical guides,
wherein at least some of the optical guides are positioned with lateral offsets with respect to the longitudinal axis;
a scanning mechanism disposed within the distal end with at least some of the optical guides of the plurality affixed to the scanning mechanism; the scanning mechanism configured to scan the affixed optical guides; and
at least one optical directing element disposed within the distal end of the first arrangement and configured to transmit the optical energy delivered by the optical guides to the tissue;
a system console including a data-processing unit with memory; the system console being in operable communication with the optical guide and the scanning mechanism and configured to:
process the optical energy acquired from the tissue by the first arrangement and delivered by the optical guide to generate image data,
process the optical energy returned by the sensing portion of the optical guide to measure strain distribution data within the sensing portion, and
calculate a position of the distal end of the first arrangement relative to a target in the tissue using the strain distribution data and a reference image data of the tissue; the reference image data pre-acquired and stored in data-processing memory of the console.

16. An image-guided system according to claim 2, wherein the position calculation further uses the image data acquired by the first arrangement.

17. An image-guided system according to claim 12, wherein the scanning mechanisms is a lateral scanning arrangement configured to scan the affixed optical guides laterally with respect to the longitudinal axis.

18. An image-guided system according to claim 12, wherein the scanning mechanisms is a torsional scanning arrangement configured to rotationally reciprocate the affixed optical guides around the longitudinal axis.

19. An image-guided system according to claim 12 further comprising a single optical directing element disposed within the distal end of the first arrangement and configured to transmit the optical energy delivered by a plurality of the optical guides to the tissue.

20. An image-guided system according to claim 12, further comprising:
a second arrangement configured to accept the first arrangement; and
the system console further configured to:
calculate a position of one of the distal end of the first arrangement and a distal end of the second arrangement relative to a target in the tissue using the strain distribution data and the reference image data of the tissue.
